(19) United States
(12) Patent Application Publication
(10) Pub. No.: US 2025/0339031 A1
(43) Pub. Date: Nov. 6, 2025

(54) SUBJECT EVALUATION DEVICE AND SUBJECT EVALUATION SYSTEM

(71) Applicant: Information System Engineering Inc., Tokyo (JP)

(72) Inventor: Satoshi KURODA, Tokyo (JP)

(21) Appl. No.: 18/871,685

(22) PCT Filed: Mar. 29, 2024
(86) PCT No.: PCT/JP2024/012989

§ 371 Date: Dec. 4, 2024

(30) Foreign Application Priority Data
Apr. 13, 2023 (JP) 2023-065985

(51) Int. Cl. A61B 5/00; A61B 5/01; A61B 5/0205; A61B 5/11

(52) U.S. Cl. CPC A61B 5/0064; A61B 5/015; A61B 5/02055; A61B 5/1116; A61B 5/7264

(57) ABSTRACT

A subject evaluation device (1) that evaluates the state of a subject (3) includes one or more sensors (2) that measure the state of the subject, an acquisition unit that acquires subject information on the subject and feature information via the sensor, a conversion unit that converts the subject information into an evaluation target image on a two-dimensional plane based on the feature information on the sensor, a reference database that stores an association between a past evaluation target image that has been preliminarily converted and reference information associated with the past evaluation target image, an evaluation unit that refers to the reference database and generates an evaluation result for the evaluation target image, and an output unit that outputs the evaluation result.

### TECHNICAL FIELD

The present invention relates to a subject evaluation device and a subject evaluation system that evaluate the state of a subject.

### BACKGROUND ART

Conventionally, for example, a determination device of Patent Document 1 has been proposed as a device that assists in evaluating the behavior of a subject.

A moving image processing device disclosed in Patent Document 1 includes an acquisition unit, an analysis unit, and a tagging processing unit. The acquisition unit acquires moving image data of a subject in a room. The analysis unit analyzes the moving image data acquired by the acquisition unit, and extracts first information on a person including the subject and second information on an object in the room. The tagging processing unit assigns a plurality of tags related to the first information and the second information extracted by the analysis unit in association with the moving image data in order to assist in evaluating a predetermined action performed by the subject. The moving image processing device efficiently assists in evaluating a predetermined action of an evaluation subject.

Patent Document 1: JP-A-2023-001531

### DISCLOSURE OF THE INVENTION

### Problems to be Solved by the Invention

Here, for example, in the moving image processing device as disclosed in Patent Document 1, it is assumed that the moving image data of the room obtained by a near-infrared camera is only analyzed. Thus, it is difficult to perform a process appropriate for a plurality of types of sensors at various locations according to on-site needs, in addition to the near-infrared camera.

Therefore, the present invention has been made in consideration of the above-described problem, and it is an object of the present invention to provide a subject evaluation device and a subject evaluation system that can perform a process appropriate for a plurality of types of sensors at various locations according to on-site needs.

### Solutions to the Problems

A subject evaluation device according to a first invention is a subject evaluation device that evaluates a state of a subject. The subject evaluation device includes one or more sensors, an acquisition unit, a conversion unit, a reference database, an evaluation unit, and an output unit. The one or more sensors measure the state of the subject. The acquisition unit acquires subject information indicating at least any of a physical condition and behavior of the subject, and feature information indicating a feature of the sensor via the sensor. The conversion unit converts the acquired subject information into an evaluation target image on a two-dimensional plane based on the feature information on the sensor. The reference database stores an association between a past evaluation target image that has been preliminarily converted and reference information associated with the past evaluation target image. The evaluation unit refers to the reference database and generates an evaluation result for the evaluation target image. The output unit outputs the evaluation result.

In the subject evaluation device according to a second invention, which is in the first invention, the association is constructed by machine learning using the past evaluation target image and the reference information as learning data.

The subject evaluation device according to a third invention, which is in the first invention or the second invention, further includes an updating unit that reflects a relationship between the past evaluation target image and the reference information in the association when the relationship is newly acquired.

A subject evaluation system according to a fourth invention is a subject evaluation system that evaluates a state of a subject. The subject evaluation system includes one or more sensors, acquisition means, conversion means, a reference database, evaluation means, and output means. The one or more sensors measure the state of the subject. The acquisition means acquires subject information indicating at least any of a physical condition and behavior of the subject, and feature information indicating a feature of the sensor via the sensor. The conversion means converts the acquired subject information into an evaluation target image on a two-dimensional plane based on the feature information on the sensor. The reference database stores an association between a past evaluation target image that has been preliminarily converted and reference information associated with the past evaluation target image. The evaluation means refers to the reference database and generates an evaluation result for the evaluation target image. The output means outputs the evaluation result.

### Effects of the Invention

According to the first invention to the third invention, the acquisition unit acquires the subject information and the feature information from the one or more sensors that measure the state of the subject. Therefore, it is possible to acquire the subject information indicating at least any of the physical condition and the behavior of the subject and the feature information indicating the feature of the sensor via a plurality of the sensors. This allows performing a process appropriate for a plurality of types of sensors at various locations according to on-site needs.

According to the first invention to the third invention, the conversion unit converts the acquired subject information into the evaluation target image on the two-dimensional plane based on the feature information on the sensor. Therefore, it is possible to refer to the reference database and generate the evaluation result for the evaluation target image. This allows performing a process appropriate for a plurality of types of sensors at various locations according to on-site needs.

In particular, according to the second invention, the association is constructed by machine learning using the past evaluation target image and the reference information as learning data. Therefore, even when an unknown evaluation target image different from the past evaluation target image is evaluated, a quantitative evaluation can be performed. This allows performing a process appropriate for a plurality of types of sensors at various locations according to on-site needs.

In particular, according to the third invention, when the updating unit newly acquires a relationship between the evaluation target image and the reference information, the updating unit reflects the relationship in the association. Therefore, even when a new evaluation target image different from the past evaluation target image is evaluated, a quantitative evaluation can be performed. This allows attempting further improvement of the evaluation accuracy, and allows performing a process appropriate for a plurality of types of sensors at various locations according to on-site needs.

According to the fourth invention, the evaluation means refers to the reference database and generates the evaluation result for the evaluation target image. The reference information includes physical information. Therefore, it is possible to generate an evaluation result based on the result of evaluating the state of the subject in the past. This allows attempting improvement of the accuracy of evaluating the state of the subject, and allows performing a process appropriate for a plurality of types of sensors at various locations according to on-site needs.

### BRIEF DESCRIPTION OF THE DRAWINGS

FIG. 1 is a schematic diagram illustrating an example of a subject evaluation system according to the embodiment.

FIG. 2A is a schematic diagram illustrating an exemplary operation of a subject evaluation device according to the embodiment at a location A. FIG. 2B is a schematic diagram illustrating an exemplary operation of the subject evaluation device according to the embodiment at a location B.

FIG. 3A is a schematic diagram illustrating an example of sensor data of the subject evaluation system according to the embodiment. FIG. 3B is a schematic diagram illustrating an example of an evaluation target image of the subject evaluation system according to the embodiment.

FIG. 4A is a schematic diagram illustrating an exemplary configuration of the subject evaluation device according to the embodiment, and FIG. 4B is a schematic diagram illustrating an exemplary function of the subject evaluation device according to the embodiment.

FIG. 5 is a schematic diagram illustrating an example of a reference database according to the embodiment.

FIG. 6 is a flowchart illustrating an exemplary operation of the subject evaluation system according to the embodiment.

### DESCRIPTION OF PREFERRED EMBODIMENTS

The following describes examples of a subject evaluation system and a subject evaluation device in an embodiment to which the present invention is applied with reference to the drawings.

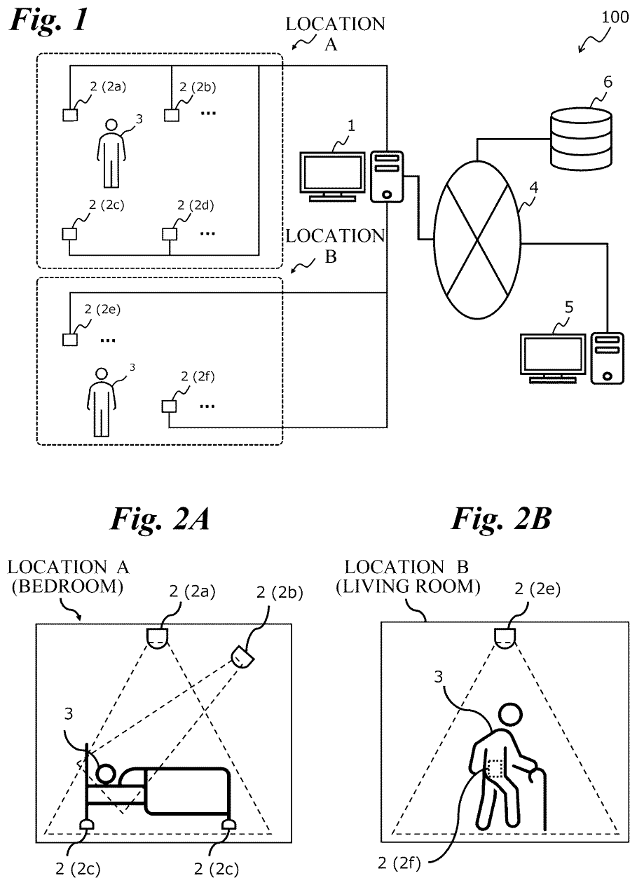

With reference to FIG. 1, examples of a subject evaluation system 100 and a subject evaluation device 1 according to the embodiment will be described.

The subject evaluation system 100 according to the embodiment includes the subject evaluation device 1, for example, as illustrated in FIG. 1. The subject evaluation device 1 is connected to, for example, sensors 2 (2a to 2f), and additionally, may be connected to another terminal 5 or a server 6 via, for example, a communications network 4.

The subject evaluation system 100 evaluates the state of a subject 3. The subject evaluation system 100 can be used for, for example, an evaluation performed after diagnosis on or observation of the subject 3 at a location A (for example, a medical examination or a medical treatment performed by a doctor or the like at a nursing home, and an observation performed by a caregiver or the like in home care).

The subject evaluation system 100 can also perform an evaluation, for example, in a situation other than medical care or nursing care. For example, the subject evaluation system 100 can be used for an evaluation of transferring, eating, bathing, medication, excretion, rehabilitation, a vital sign check, sputum suction, respiratory support, a drip infusion, a blood transfusion, and the like for the subject 3, and further, can also be used for an evaluation performed on the state of the subject 3 at the time of work in various on-site situations, such as delivery work at a logistics site, assembly work at a manufacturing site, and sales work at a sales site.

Further, the subject evaluation system 100 may evaluate the future state based on the current state of the subject 3. The subject evaluation system 100 can also be used for evaluations, for example, “no problem with home care since the condition has recovered” based on an evaluation performed after diagnosis on the subject 3 at a nursing home, and “nursing care at a nursing home is likely to be required” based on an evaluation performed after observation of the subject 3 in home care.

Here, with reference to FIG. 2, an exemplary operation of the subject evaluation device 1 at the location A will be described. First, FIG. 2A illustrates an exemplary operation of the subject evaluation device 1 targeted at the location A (a bedroom), for example. As illustrated in FIG. 2A, the subject evaluation device 1 uses one or more sensors 2 (2a to 2f) that measure the state of the subject 3 while sleeping, for example, to measure data on the state of the subject 3 while sleeping.

In the sensors 2, for example, the sensor 2a is a motion sensor that measures the movement of the subject 3, which measures and quantifies the measured movement of the subject 3. For example, the sensor 2b is a near-infrared or non-contact vital sensor that measures a body temperature or vital signs of the subject 3, which measures and quantifies the measured body temperature or vital signs of the subject 3. For example, the sensor 2c is a near-infrared or non-contact vital sensor that measures the posture, the center of gravity, and the like of the subject 3, which measures and quantifies the measured body temperature or vital signs of the subject 3. For example, the sensors 2c are sensors that are attached to legs of a bed or the like and measure the orientation and the posture of the body and the center of gravity of the body of the subject 3, which measure and quantify the measured orientation and posture of the body and center of gravity of the body of the subject 3.

The subject evaluation device 1 acquires various kinds of measurement data measured by the respective sensors 2 (2a to 2c). For example, when the various kinds of measurement data measured by the respective sensors 2 (2a to 2c) are acquired, the subject evaluation device 1 acquires feature information (for example, a sensor ID, a measurement data ID, a measurement data characteristic, a measurement date and time, a measurement location, and the like) indicating features of the respective sensors together. The subject evaluation device 1 may, for example, preliminarily set an acquisition condition, such as a transmission date and time to the subject evaluation device 1 and measurement data to be transmitted, for the respective sensors 2 (2a to 2c), and the respective sensors 2 may transmit the measurement data to the subject evaluation device 1 based on the set acquisition condition.

Next, FIG. 2B illustrates an exemplary operation of the subject evaluation device 1 targeted at a location B (a living room), for example. As illustrated in FIG. 2B, for example, the subject evaluation device 1 measures data on a behavior state of the subject 3 in which the subject 3 gets up and moves from the location A, using one or more sensors 2 (2e to 2f).

In the sensors 2, for example, the sensor 2e is a motion sensor that measures the movement of the subject 3, which measures and quantifies the measured movement of the subject 3. For example, the sensor 2f is a wearable sensor worn by the subject 3, which measures and quantifies the number of steps, the movement of hands and feet, the heart rate, the respiratory rate, and the like of the subject 3.

A plurality of the sensors 2 (2a to 2f) illustrated in FIGS. 2A and 2B may be installed, for example, in a facility or at home. As installation sites, for example, the sensors 2 may be installed on a ceiling, a wall, a table, a bed, a chair, a wheelchair, an automobile, or the like, or may be worn by an evaluator. When the installation place is a bathroom, for example, sensor data may be acquired by a near-infrared camera or the like without acquiring image data of the body state of the subject 3. This allows acquiring the state of the subject 3 by a plurality of types of sensors on site or at various locations and the like according to on-site needs.

The subject evaluation device 1 acquires various kinds of measurement data measured by the respective sensors 2 (2e to 2f). For example, when the various kinds of measurement data measured by the respective sensors 2 (2e to 2f) are acquired, the subject evaluation device 1 acquires feature information (for example, a sensor ID, a measurement data ID, a measurement data characteristic, a measurement date and time, a measurement location, and the like) indicating features of the respective sensors together. The subject evaluation device 1 may, for example, preliminarily set an acquisition condition, such as a transmission date and time to the subject evaluation device 1 and measurement data to be transmitted, for the respective sensors 2 (2e to 2f), and the respective sensors 2 may transmit the measurement data to the subject evaluation device 1 based on the set acquisition condition.

The subject evaluation device 1 acquires subject information indicating at least any of the physical condition and the behavior of the subject 3 and feature information indicating the feature of the sensor 2 via the sensor 2, and converts the acquired subject information into an evaluation target image on a two-dimensional plane based on the feature information on the sensor.

The subject evaluation system 100 refers to, for example, a reference database in which an association between a past evaluation target image that has been preliminarily converted and reference information associated with the past evaluation target image is stored, and generates an evaluation result for the evaluation target image. Thus, the state of the subject 3 can be evaluated using the current evaluation target image of the subject 3. For example, a plurality of evaluation target images may be combined to be evaluated. From the plurality of evaluation target images, for example, according to a combination of characteristic evaluation target images, various kinds of states of the subject 3 can be checked or a future occurrence can be predicted.

After acquiring evaluation target information including the measurement data, with an output unit that outputs the evaluation result, the subject evaluation system 100 refers to the reference database described later and generates the evaluation result for the evaluation target image. The subject evaluation device 1 outputs the generated evaluation result to a display unit 109 or the like.

The evaluation result indicates, in addition to the current confirmed state with the state of the subject 3 as an evaluation target, a prediction result of an unconfirmed state that may occur or be developed in the future, and the like. The evaluation result indicates the evaluation result of the state related to the body or the behavior of the subject 3, for example, “normal,” “abnormal,” and “observation required.” Additionally, the evaluation result may indicate tendencies of a possible case and condition, necessary nursing care, and the like for the subject 3, and probabilities of a medical state and a condition that may be developed in the future, necessary nursing care, and the like, for example, “tendency to have XX symptom” and “probability of XX symptom 60%.” This allows, for example, a caregiver of the subject 3 to prepare for the future nursing care and care of the subject 3 and the like in advance by confirming these evaluation results.

As the subject evaluation device 1, an electronic device, such as a personal computer (PC), is used, and additionally, an electronic device, such as a smartphone, a tablet terminal, a wearable device, and an IoT (Internet of Things) device, or a single board computer, such as Raspberry Pi (registered trademark), may be used, and for example, the subject evaluation device 1 may include the built-in sensor 2. For example, when a head mounted display (HMD) including the built-in sensor 2 is used as the subject evaluation device 1, the evaluator or the like can recognize the evaluation result of the subject 3 by visually recognizing the subject 3 through the display. Therefore, the difficulty of body evaluation work for the subject 3 can be decreased, and moreover, an evaluation work time can be shortened.

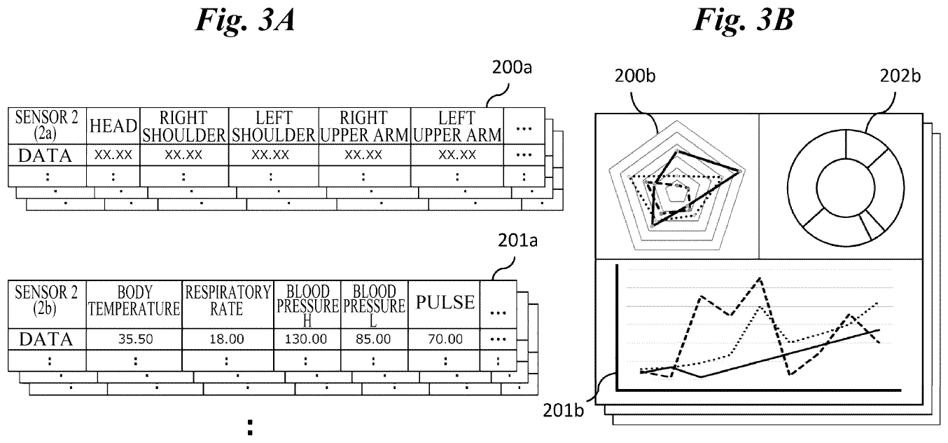

Here, with reference to FIG. 3, an example of sensor data of the subject evaluation system 100 according to the embodiment will be described. FIG. 3A illustrates an example of sensor data of the subject evaluation system 100 according to the embodiment, and various kinds of sensor data measured by the plurality of sensors 2 are acquired by the subject evaluation device 1.

As the sensor data measured by the various sensors 2, for example, the sensor 2a measures the state of the subject 3 (for example, behavior information during sleep and the like) and records the state as sensor data 200a. As the sensor data 200a, for example, coordinates of respective positions and movements such as the position of the head (XX. XX), the position of the right shoulder (XX. XX), the position of the left shoulder (XX. XX), the position of the right upper arm (XX. XX), and the position of the left upper arm (XX. XX) of the subject 3, and numerical values indicating actions are measured in chronological order, and a plurality of the measured sensor data 200a are recorded.

The sensor 2a measures and quantifies the movement of the subject 3 by, for example, a publicly known motion sensor or the like. The sensor data 200a may be measured using, for example, a near-infrared sensor, an image sensor, or the like (not illustrated) in addition to the publicly known motion sensor, and is acquired using a publicly known measurement technique.

The sensor data 200a measured by the sensor 2a is acquired by, for example, the subject evaluation device 1, and a plurality of the sensor data 200a are recorded in the subject evaluation device 1 as the sensor data 200a, and additionally, the sensor data 200a may be recorded in, for example, the other terminal 5, the server 6, or the like.

For example, the sensor 2b measures the state of the subject 3 (for example, vital information during sleep and the like) and records the state as sensor data 201a. As the sensor data 201a, for example, numerical values indicating respective pieces of the vital information, such as the body temperature (35.50), the respiratory rate (18.00), the blood pressure value High (130.00), the blood pressure value Low (85.00), and the pulse rate (70.00) of the subject 3, are measured in chronological order, and a plurality of the measured sensor data 201a are recorded.

The sensor 2b measures and quantifies the vital information on the subject 3 using, for example, a publicly known near-infrared or non-contact vital sensor or the like. The sensor data 201a may be measured using, for example, various image sensors or the like (not illustrated) in addition to a publicly known vital sensor, and is acquired using a publicly known measurement technique.

The sensor data 201a measured by the sensor 2b is acquired by, for example, the subject evaluation device 1, and a plurality of the sensor data 201a are recorded in the subject evaluation device 1 as the sensor data 201a, and additionally, the sensor data 201a may be recorded in, for example, the other terminal 5, the server 6, or the like.

In addition to the sensor data 200a by the sensor 2a and the sensor data 201a by the sensor 2b, sensor data by each of the other sensors 2c to 2f is similarly measured, and a plurality of the sensor data are recorded in the subject evaluation device 1 as the respective sensor data, and additionally, the sensor data is recorded in, for example, the other terminal 5, the server 6, or the like.

FIG. 3B illustrates an example of the evaluation target image of the subject evaluation system 100 according to the embodiment, in which various kinds of sensor data measured by the sensors 2 are converted into evaluation target images 200b to 202b on the two-dimensional plane based on feature information (the type, model, conversion parameter, performance/property, visualization property, format, and the like) on the respective sensors 2.

The evaluation target image 200b is obtained by, for example, converting the sensor data 200a into the evaluation target image 200b on the two-dimensional plane based on the feature information on the sensor 2a. The image conversion is, for example, a known graphing process or the like, and refers to a state in which the sensor data measured in chronological order is graphed based on a specific parameter or the like.

While the evaluation target image 200b is obtained by converting the numerical values measured by the sensor 2a into the evaluation target image 200b on the two-dimensional plane with a radar chart, the numerical values may be converted into a graph other than the radar chart when other features are to be represented.

Similarly, the evaluation target image 201b is obtained by, for example, converting the sensor data 201a into the evaluation target image 201b on the two-dimensional plane based on the feature information on the sensor 2b, and for example, obtained by converting changes in the respective pieces of the vital information in chronological order into the evaluation target image 201b on the two-dimensional plane.

The evaluation target image 202b may be obtained by, for example, converting the sensor data measured by another sensor 2 into the evaluation target image 202b on the two-dimensional plane based on the feature information on the sensor 2. Alternatively, the respective numerical values measured by the plurality of sensors 2 may be compiled, and the proportion of the numerical values and the like may be converted into the evaluation target image 201b on the two-dimensional plane.

There may be a plurality of evaluation target images illustrated in FIG. 3B for the respective sensors 2a to 2f, and further, when the measurement is performed in chronological order, a time-series transition or numerical values summed during a certain period of time may be converted into an image as a proportion.

The subject evaluation system 100 displays the evaluation target images 200b to 202b and a plurality of other evaluation target images on the two-dimensional plane that have been converted on the display unit 109. For example, when the plurality of the sensor data are measured by one sensor 2, the subject evaluation device 1 can separately generate an evaluation result for the subject 3, and the display unit 109 can display the evaluation result for the subject 3. For example, when an evaluation result for one subject 3 is generated, the evaluation result may be based on the plurality of sensor data. For example, the type and the number of the plurality of evaluation target images to be combined and displayed are appropriately set.

The sensor data may be generated using, for example, an RGB camera or the like. The sensor data may be generated using, for example, a multispectral camera with any wavelength selected, and may be generated, for example, based on imaging via a polarizing filter. For example, the sensor data may be extracted from a part of a moving image, and may be converted into an image as an evaluation target image.

The subject information is, for example, directly input to the subject evaluation device 1 by the evaluator or the like so as to be associated with the sensor data acquired by the subject evaluation device 1, and additionally, for example, a plurality of pieces of the subject information may be preliminarily stored in the subject evaluation device 1 and selected by the subject evaluation device 1 based on the evaluation target image. When the subject evaluation device 1 selects the subject information, for example, the subject or the sensor 2 may be selected for the acquired evaluation target image using a learning model that is preliminarily stored in the subject evaluation device 1. In this case, the learning model is generated by publicly known machine learning that uses the evaluation target image and the subject information prepared in advance as learning data.

The subject information includes information on at least any of a date and time, a posture, and an action when the subject 3 is observed, which are measured as sensor data, an observer, and a date and time when the subject 3 is to be observed. The subject information may include, for example, information on a daily life of the subject 3, such as physical strength, training, and meals.

The subject information is, for example, directly input to the subject evaluation device 1 by the evaluator or the like so as to be associated with the sensor data acquired by the subject evaluation device 1, and additionally, may be transmitted from, for example, the other terminal 5 or the like.

### Subject Evaluation Device 1

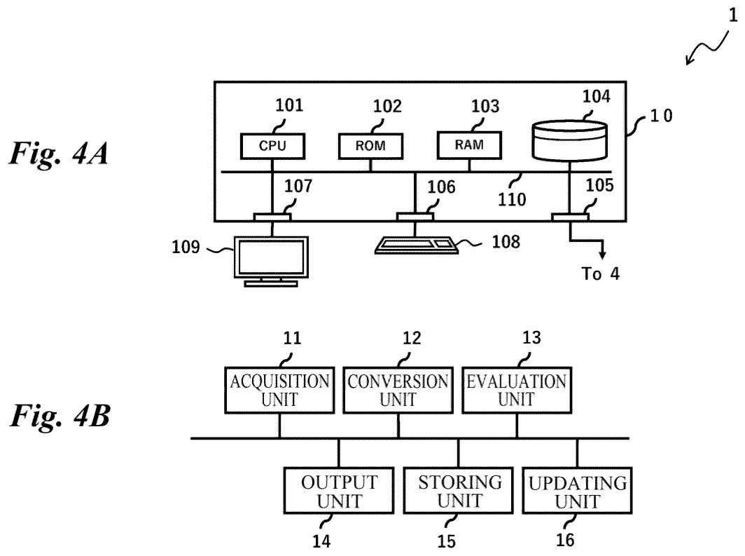

Next, with reference to FIG. 4, an example of the subject evaluation device 1 according to the embodiment will be described. FIG. 4A is a schematic diagram illustrating an exemplary configuration of the subject evaluation device 1 according to the embodiment, and FIG. 4B is a schematic diagram illustrating an exemplary function of the subject evaluation device 1 according to the embodiment.

For example, as illustrated in FIG. 4A, the subject evaluation device 1 includes a housing 10, a central processing unit (CPU) 101, a read only memory (ROM) 102, a random access memory (RAM) 103, a storage unit 104, and I/Fs 105 to 107. The respective components 101 to 107 are connected by an internal bus 110.

The CPU 101 controls the whole of the subject evaluation device 1. The ROM 102 stores an operation code of the CPU 101. The RAM 103 is a work area used in the operation of the CPU 101. The storage unit 104 stores various kinds of information, such as the evaluation target image and the reference database. As the storage unit 104, for example, in addition to a hard disk drive (HDD), a data storage device, such as a solid state drive (SSD), is used. For example, the subject evaluation device 1 may include a graphics processing unit (GPU) (not illustrated). Including the GPU allows faster arithmetic processing than usual.

The I/F 105 is an interface for transmitting and receiving various kinds of information with the sensor 2, and additionally, for example, may be an interface for transmitting and receiving various information with the other terminal 5, the server 6, or the like via the communications network 4, such as the Internet.

The I/F 106 is an interface for transmitting and receiving information with an input unit 108. As the input unit 108, for example, a keyboard is used, and the evaluator or the like using the subject evaluation device I inputs various kinds of information or a control command or the like of the subject evaluation device 1 via the input unit 108.

The I/F 107 is an interface for transmitting and receiving various kinds of information with the display unit 109. The display unit 109 outputs various kinds of information, such as an evaluation result, stored in the storage unit 104, a process status of the subject evaluation device 1, or the like. As the display unit 109, a display is used, and for example, a touch panel type may be used.

### Reference Database

In the reference database stored in the storage unit 104, an association between a preliminarily acquired past evaluation target image and reference information associated with the past evaluation target image is stored, and for example, a learning model having the association is stored. The reference database may store, for example, the past evaluation target image and the reference information. The association is constructed, for example, by machine learning using a plurality of learning data with the past evaluation target image and the reference information as a set of learning data. As a learning method, for example, deep learning, such as a convolutional neural network, is used.

In this case, for example, the association indicates the degree of connection between many-to-many pieces of information (a plurality of data included in the past evaluation target image versus a plurality of data included in the reference information). The association is appropriately updated in the course of machine learning. That is, the association indicates a function that is optimized based on, for example, the past evaluation target image (image data on the two-dimensional plane) and the reference information. Therefore, the evaluation result for the evaluation target image is generated using the association constructed based on all the results obtained by evaluating the state of the subject 3 in the past. This allows generating an optimal evaluation result even when the physical condition and the behavior of the subject 3 are in various states.

The optimal evaluation result can be quantitatively generated not only when the evaluation target image is identical or similar to the past evaluation target image but also when the evaluation target image is dissimilar to the past evaluation target image. The improvement of evaluation accuracy for an unknown evaluation target image can be attempted by increasing generalization capability when machine learning is performed.

The association may include, for example, a plurality of association degrees indicating the degree of connection between a plurality of data included in the past evaluation target image and a plurality of data included in the reference information. For example, the association degree can correspond to a weight variable when the learning model is constructed with a neural network.

The past evaluation target image indicates the same kind of information as the above-described evaluation target image. The past evaluation target image includes, for example, a plurality of evaluation target images acquired when the subject 3 was evaluated in the past.

The reference information is associated with the past evaluation target image, and indicates information on the state of the subject 3. The reference information indicates an evaluation based on the state of the subject 3 (for example, “normal,” “abnormal,” “observation required, “tendency to have XX symptom,” “probability of XX symptom 60%,” and the like). Additionally, the reference information may include physical information, correspondence information, preparation information, predictive information, and the like related to a factor of the state of the subject 3.

The reference information may indicate, for example, tendencies of a possible case and condition, necessary nursing care, and the like for the subject 3, and probabilities of a medical state and a condition that may be developed in the future, necessary nursing care, and the like. The specific contents included in the reference information can be appropriately set.

In the case of an elderly person, the physical information indicates a name of a factor of a specific state such as dementia, dehydration, gait disturbance, psychological disturbance, mobility impairment, impaired excretory function, sensory disturbance, and nutritional disturbance. The various factors are generally related to at least a part of the physical condition and the behavior of the subject 3.

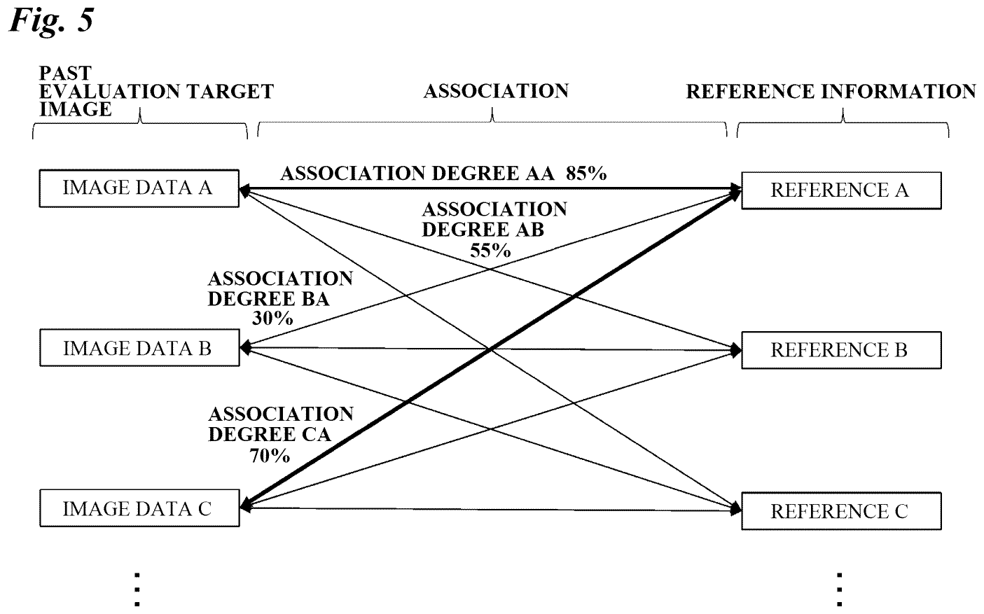

For example, as illustrated in FIG. 5, the association may indicate the degree of connection between the past evaluation target image and the reference information. In this case, by using the association, a plurality of data (in FIG. 5, “Image Data A” to “Image Data C”) included in the past evaluation target image can be each stored in association with the degree of relationship with a plurality of data (in FIG. 5, “Reference A” to “Reference C”) included in the reference information. Therefore, for example, one piece of data included in the past evaluation target image can be associated with a plurality of data included in the reference information via the association, and thus the generation of a multifaceted evaluation result can be achieved.

For example, the association includes a plurality of association degrees associating a plurality of data included in the past evaluation target image with a plurality of data included in the reference information respectively. The association degree is indicated by, for example, a percentage, or three or more levels, such as ten levels or five levels, and indicated by, for example, the feature (for example, the thickness or the like) of a line. For example, the “image data A” included in the past evaluation target image has an association degree AA of “85%” with the “reference A” included in the reference information, and has an association degree AB of “55%” with the “reference B” included in the reference information. That is, the “association degree” indicates the degree of connection between the respective image data, and for example, it is indicated that the connection between the respective data becomes stronger as the association degree increases. When the association is constructed by the above-described machine learning, the association may be set to have the association degree of three or more levels.

The past evaluation target image may be stored in the reference database, for example, by dividing the past image data A to C and the past physical condition information or behavior information. In this case, the association degree is calculated based on a relationship between a combination of the past image data and the past physical condition information or behavior information and the reference information. For example, in addition to the above, the past evaluation target image may be stored in the reference database by dividing the past observation information (various kinds of sensor data and the like).

The past evaluation target image may include, for example, composite data and the degree of similarity. The composite data is indicated by three or more levels of the degree of similarity to the past image data, or the past physical condition information or behavior information. The composite data is stored in the reference database in the format of a numerical value, a matrix, a histogram, or the like, and additionally, may be stored in the format of, for example, an image, a character string, or the like.

FIG. 4B is a schematic diagram illustrating an exemplary function of the subject evaluation device 1. The subject evaluation device 1 includes an acquisition unit 11, a conversion unit 12, an evaluation unit 13, and an output unit 14, and may have, for example, an updating unit 16. The respective functions illustrated in FIG. 4B are achieved by executing a program stored in the storage unit 104 or the like by the CPU 101 with the RAM 103 as a work area, and may be controlled by, for example, artificial intelligence.

### Acquisition Unit 11

The acquisition unit 11 acquires the subject information indicating at least any of the physical condition and the behavior of the subject 3 and the feature information indicating the feature of the sensor 2 via one or more sensors that measure the state of the subject 3. The acquisition unit 11 acquires sensor data indicating the subject information on the subject 3 from the sensor 2 or the like, and additionally, for example, when a built-in camera is included, the acquisition unit 11 may acquire image data of the subject 3, spatial image data of a location, and the like from the sensor 2. The acquisition unit 11 acquires the physical condition information and the behavior information on the subject 3 input in advance by the evaluator or the like, and additionally, for example, acquires the feature information for identifying the sensor 2 from the sensor 2 or the like.

For example, when acquiring the sensor data, the acquisition unit 11 acquires the feature information on the sensor 2 together with the sensor data. The frequency and the cycle of acquiring the subject information and the feature information by the acquisition unit 11 are appropriately set.

The acquisition unit 11 receives various kinds of information including the sensor data on the evaluator measured by the sensor 2 and the feature information. The acquisition unit 11 may, for example, receive various kinds of information, such as the physical condition information, the behavior information, the observation information, and environmental information on a location of the subject 3, transmitted from an external terminal such as the other terminal 5 via the communications network 4 and the I/F 105.

For example, the acquisition unit 11 may refer to the learning model stored in the storage unit 104, select the physical condition information and the behavior information corresponding to the sensor 2, the sensor data, and the feature information, and acquire them as an evaluation target image.

### Conversion Unit 12

The conversion unit 12 converts the subject information acquired by the acquisition unit 11 into the evaluation target image on the two-dimensional plane based on the feature information on the sensor 2. As illustrated in FIG. 3, for example, the conversion unit 12 converts the sensor data 200a measured by the various sensors 2a into the evaluation target image 200b. The conversion unit 12 performs an image conversion into a graph (for example, a radar chart) as illustrated in FIG. 3B based on, for example, various kinds of information (for example, the type, model, conversion parameter, performance/property, visualization property, format, and the like), which is sensor property information on the sensor 2a or property information on the sensor data.

For example, the conversion unit 12 performs an image conversion by graphing based on the position of the head (XX. XX), the position of the right shoulder (XX. XX), the position of the left shoulder (XX. XX), the position of the right upper arm (XX. XX), and the position of the left upper arm (XX. XX) as items indicating the state of the subject 3 acquired by the sensor 2a, and numerical values for each item. For example, the conversion unit 12 may refer to a correspondence table (not illustrated) for performing graphing, graph the sensor data 200a measured by the sensor 2a, and perform an image conversion.

For example, when the measured sensor data 200a has a partial feature, the conversion unit 12 may graph only the range and numerical values of the partial feature. The conversion unit 12 may graph the sensor data on the target sensor 2 and perform an image conversion based on, for example, instructions from the other terminal 5. The conversion unit 12 performs a similar image conversion for the sensor 2b and the other sensors 2, for example. The conversion unit 12 may combine the numerical value data measured by the plurality of sensors 2 to generate a new graph, for example, and may use the combined graph as an evaluation target image.

For example, the conversion unit 12 may compile the respective numerical values measured by the plurality of sensors 2, graph the proportion and the transition of the numerical values and the like, and perform an image conversion. For example, when the sensor data is measured in chronological order and there is a tendency of a significant increase or decrease during a certain period of time, the conversion unit 12 may graph the tendency and store a condition and a parameter such as a period of time and a numerical value at the time of graphing, various kinds of setting information at the time of graphing, and the like together. This allows the evaluation target image to be checked back to the graphed period of time, and an image conversion process appropriate for a plurality of types of sensors can be performed at various locations according to on-site needs.

### Evaluation Unit 13

The evaluation unit 13 refers to the reference database and generates the evaluation result for the evaluation target image. The evaluation unit 13, for example, uses the evaluation target image as input data, selects the optimal reference information associated with a solution calculated based on the association, and generates the evaluation result based on the optimal reference information.

For example, when the evaluation unit 13 refers to the reference database illustrated in FIG. 5, the evaluation unit 13 selects data (for example, the “image data A”: first data) identical or similar to the data included in the evaluation target image. As the first data, image data that partially matches or exactly matches the evaluation target image is selected, and additionally, for example, similar image data is selected. When the evaluation target image is represented by a numerical value, such as a matrix, the numerical value range included in the first data to be selected may be preliminarily set.

The evaluation unit 13 selects the reference information associated with the selected first data and the association degree (a first association degree) between the selected first data and the reference information, and generates an evaluation result based on the selected reference information and the first association degree. The first association degree is selected from a preliminarily constructed association, and additionally, may be calculated by the evaluation unit 13.

For example, the evaluation unit 13 selects the data “reference A” included in the reference information associated with the first data “image data A” and the first association degree (the association degree AA) “85%” between the “image data A” and the “reference A.” The reference information and the first association degree may include a plurality of data. In this case, in addition to the above-described “reference A” and “85%,” the reference information “reference B” associated with the first data “image data A” and the first association degree (the association degree AB) “55%” between the “image data A” and the “reference B” may be selected, and the evaluation result may be generated based on the “reference A” and “85%,” and the “reference B” and “55%.”

The evaluation result may include the evaluation target image. The evaluation result may, for example, indicate a factor of the state (the physical condition or the behavior) of the subject 3 that is represented by a probability using the reference information and the association degree.

The evaluation unit 13 generates, for example, an evaluation result indicating the selected reference information, the first association degree, and the like described above in a format (for example, a character string) that can be understood by the evaluator or the like, using format data such as an output format that is preliminarily stored in the storage unit 104 or the like. For example, a publicly known technique may be used for setting of the format or the like in generating the evaluation result.

The evaluation unit 13 determines the contents of the evaluation result based on, for example, the selected first association degree. For example, the evaluation unit 13 may be configured to generate the evaluation result based on the reference information associated with the first association degree of “50%” or more, and not to reflect the reference information associated with the first association degree of less than “50%” in the evaluation result. For a determination criterion based on the first association degree, for example, the evaluator or the like may preliminarily set a threshold value or the like, and the range of the threshold value or the like can be appropriately set. The evaluation unit 13 may determine the contents of the evaluation result based on, for example, a result of calculating two or more first association degrees or a comparison of two or more first association degrees.

### Output Unit 14

The output unit 14 outputs the evaluation result. The output unit 14 transmits the evaluation result to the display unit 109 via the I/F 107, and additionally, for example, transmits the evaluation result to the other terminal 5 and the like via the I/F 105. The output unit 14 outputs, for example, the evaluation target image of the subject 3 illustrated in FIG. 3 and data indicating the optimal evaluation result, recommendation, and the like corresponding to the physical condition and the behavior of the subject 3, to the display unit 109 or the like.

The output unit may display, for example, the result of the evaluation based on the state of the subject 3 (for example, “normal,” “abnormal,” “observation required,” “tendency to have XX symptom,” “probability of XX symptom 60%,” and the like). Additionally, the output unit may also display, for example, the physical information, the correspondence information, the preparation information, the predictive information, and the like related to the factor of the current or future state of the subject 3.

### Storing Unit 15

A storing unit 15 takes out various kinds of information, such as the reference database, stored in the storage unit 104 as necessary. The storing unit 15 stores the various kinds of information acquired or generated by each of the components 11 and 13 to 15 in the storage unit 104.

### Updating Unit 16

For example, when the updating unit 16 newly acquires a relationship between the past evaluation target image and the reference information, the updating unit 16 reflects the relationship in the association. For example, when the subject evaluation device 1 acquires a determination result in which the evaluator or the like determines the accuracy of the evaluation result based on the evaluation result generated by the evaluation unit 13, the updating unit 16 updates the association stored in the reference database based on the determination result.

### Display Unit 109

The display unit 109 displays the evaluation result. For example, as illustrated in FIG. 3, the display unit 109 displays the evaluation target images 200b to 202b and the evaluation result. The evaluation target images 200b to 202b display the evaluation target image obtained by converting the subject information acquired by the acquisition unit 11 via the sensor 2 into the evaluation target image on the two-dimensional plane based on the feature information indicating the feature of the sensor 2, and the evaluation result, using the subject information and the feature information on the sensor 2.

The display unit 109 may display the evaluation result using, for example, only a list or a character string. A publicly known technique can be used for the above-described display method. For example, when an HMD is used as the subject evaluation device 1, a transmissive display is used as the display unit 109. At this time, for example, the display unit 109 can display the evaluation target image and the evaluation result to the subject 3 visually recognized by the evaluator or the like through the display unit 109.

### Updating Unit 16

The updating unit 16, for example, updates the reference database. When the updating unit 16 newly acquires the relationship between the past evaluation target image and the reference information, the updating unit 16 reflects the relationship in the association. For example, when the evaluator or the like determines the accuracy of the contents of the evaluation result based on the evaluation result generated by the evaluation unit 13, and the subject evaluation device 1 acquires the determination result, the updating unit 16 updates the association included in the reference database based on the determination result.

### Sensor 2

The sensors 2 are various publicly known sensors that measure the physical condition and the state of the behavior of the subject 3 and generate the sensor data. As the sensor 2, for example, various sensors such as a motion sensor, a near-infrared camera, an RGB camera, an ultrasonic sensor, and a discrimination site sensor may be used, and a plurality of sensors may be used simultaneously or in cooperation at different locations. For example, the sensor 2 may be built in the subject evaluation device 1, or may be held or worn by the subject 3.

### Communications Network 4

The communications network 4 is, for example, an Internet network to which the subject evaluation device 1, the plurality of sensors 2, or the like is connected via a communication circuit. The communications network 4 may be configured by what is called an optical fiber communications network. The communications network 4 may be achieved by a publicly known communications network, such as a wireless communications network, in addition to a wired communications network.

### Another Terminal 5

As the other terminal 5, for example, a terminal embodied in an electronic device, similarly to the subject evaluation device 1, is used. The other terminal 5 refers to, for example, a central control unit capable of communicating with a plurality of the subject evaluation devices 1. The other terminal 5 can be connected to, for example, the plurality of subject evaluation devices 1, and can acquire the evaluation results generated by each subject evaluation device 1. This allows, for example, analyzing the evaluation results of the subject 3 measured at multiple locations, and thus attempting improvement of the body state of the subject 3 or the like.

### Server 6

The server 6 stores, for example, the above-described various kinds of information. The server 6 accumulates, for example, various kinds of sensor data and various kinds of information on the plurality of sensors 2 transmitted via the communications network 4. For example, the server 6 may store information similar to the storage unit 104, and may perform transmitting and receiving various kinds of sensor data, various kinds of information on the plurality of sensors 2, image data, evaluation results, and the like with one or more subject evaluation devices 1 via the communications network 4. That is, in the subject evaluation device 1, the server 6 may be used instead of the storage unit 104.

### Exemplary Operation of Subject Evaluation System 100

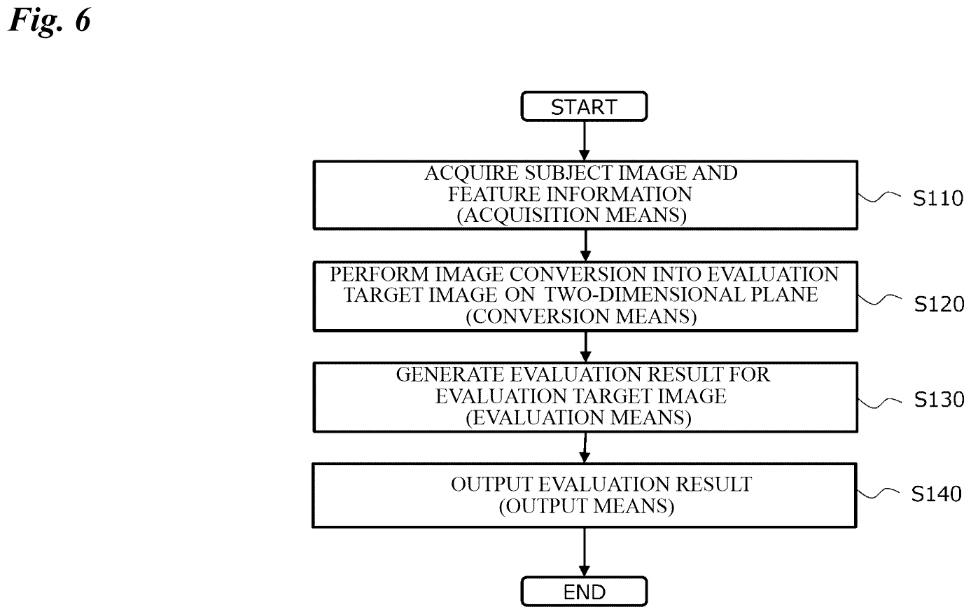

Next, an exemplary operation of the subject evaluation system 100 according to the embodiment will be described. FIG. 6 is a flowchart illustrating the exemplary operation of the subject evaluation system 100 according to the embodiment.

### Acquisition Means S110

As illustrated in FIG. 6, a subject image and the feature information are acquired (acquisition means S110). The acquisition unit 11 acquires, for example, from one or more sensors 2 that measure the state of the subject 3, the subject information indicating at least any of the physical condition and the behavior of the subject 3 and the feature information indicating the feature of the sensors 2 via the sensors 2. The acquisition unit 11 stores the evaluation target information and the feature information in the storage unit 104, for example, via the storing unit 15.

The acquisition unit 11 acquires, for example, the subject information indicating at least any of the physical condition and the behavior of the subject 3 and the feature information indicating the feature of the sensor 2. The acquisition unit 11 may be, for example, the plurality of sensors 2. The subject information and the feature information are input to the subject evaluation device 1 by the evaluator or the like so as to be associated with the image data or the like, and additionally, for example, the acquisition unit 11 may select each piece of information appropriate for the image data based on the image data. In this case, the acquisition unit 11 selects each piece of information appropriate for the image data from information (sensor data and the like) obtained by observing a plurality of subjects 3 that is preliminarily stored in the storage unit 104.

For example, when one subject 3 is imaged using the plurality of sensors 2, the acquisition unit 11 acquires the plurality of the sensor data (subject information) measured by the plurality of sensors 2 and the feature information as conversion source data to be converted into one evaluation target image. The acquisition unit 11 acquires the sensor data (subject information and feature information) indicating the state of the subject 3 in accordance with the physical condition and the behavior pattern of the evaluator, and additionally, may acquire the sensor data in accordance with, for example, patterns of life and behavior of the subject 3.

The acquisition unit 11 may, for example, convert the sensor data of the sensor 2 measured during any period of time into the acquired evaluation target image on the two-dimensional plane such that the sensor data is received at one time. When the feature information on each sensor 2 has already been acquired by the subject evaluation device 1 in advance and there is no change, for example, only the sensor data on the subject information may be acquired from the sensor 2. In this case, the subject evaluation device 1 may determine the sensor data acquired from the sensor 2 and separately acquire the feature information on the sensor 2 associated with the sensor data.

### Conversion Means S120

Next, the conversion unit 12 performs an image conversion into the evaluation target image on the two-dimensional plane (conversion means S120). For example, the conversion unit 12 converts the subject information (sensor data) on the subject 3 acquired by the acquisition unit 11 via the sensor 2 into the evaluation target image visualized as a graph on the two-dimensional plane based on the feature information on the corresponding sensor 2. For example, the conversion unit 12 converts the subject information acquired by the acquisition unit 11 into a graph or the like as an evaluation target image on the two-dimensional plane based on the feature information on the plurality of sensors 2.

The conversion unit 12 performs an image conversion into a graph as a target image on the two-dimensional plane, but may also perform an image conversion into, for example, an image other than a graph. The evaluation target image to be converted by the conversion unit 12 may be any two-dimensional planar image of the state of the subject 3, and the type and the scale of the graph, the number of graphs, the representation of the graph, and the like are appropriately set.

### Evaluation Means S130

Next, the evaluation unit 13 refers to the reference database and performs an image conversion into the evaluation target image on the two-dimensional plane (evaluation means S130). The evaluation unit 13 acquires the evaluation target image acquired by the acquisition unit 11, and for example, acquires the evaluation target image from the reference database stored in the storage unit 104. For example, the evaluation unit 13 selects the optimal reference information associated with the solution calculated based on the association indicated by the function or the like, using the evaluation target image as input data, and generates the evaluation result based on the optimal reference information. At this time, for example, the evaluation unit 13 may select a plurality of pieces of the reference information for one evaluation target image.

The evaluation unit 13 generates one evaluation result for one evaluation target image, and additionally, may generate, for example, one evaluation result for a plurality of evaluation target images. The evaluation unit 13 generates the evaluation result using, for example, the format data such as the output format stored in the storage unit 104. The evaluation unit 13 stores the evaluation result in the storage unit 104, for example, via the storing unit 15.

### Output Means S140

Next, the output unit 14 outputs the evaluation result (output means S140). The output unit 14 outputs the evaluation result to the display unit 109 or the like. The output unit 14 may output the evaluation result to the other terminal 5 or the server 6, for example, via the communications network 4.

For example, the output unit 14 may output, to the display unit 109, information for causing the display unit 109 to display the following: the subject 3 based on the image data; the sensor 2 that can measure the subject information on the subject 3; a display image showing the sensor data measured by the sensor 2, a sensing status and a sensing result of the sensor 2, a plurality of evaluation target images, the evaluation result of the subject 3 by a combination of the evaluation target images, the recommendation information based on the evaluation result, and the like; a designation unit (not illustrated) that designates the subject 3 to be evaluated in the display image; and the evaluation result for the subject 3 to be evaluated via the designation unit. This causes the display unit 109 to display the display image, the designation unit, and the evaluation result.

This terminates the operation of the subject evaluation system 100 according to the embodiment. The timing at which the updating unit 16 performs an update is appropriately set.

According to the embodiment, the evaluation unit 13 refers to the reference database and generates the evaluation result for the evaluation target image. The reference information includes various pieces of physical information and behavior information on the subject 3. Therefore, it is possible to generate the evaluation result based on the result of evaluating the state of the subject 3 in the past. This allows attempting improvement of the accuracy of evaluating the state of the subject 3, and allows performing a process appropriate for a plurality of types of sensors at various locations according to on-site needs.

According to the embodiment, the evaluation target image includes the physical condition information. Therefore, it is possible to achieve an evaluation based on the feature of the factor of the state that is different depending on the sensor 2 and the physical condition of the subject 3 and the like. This allows further improvement of the accuracy of evaluating the state of the subject 3, and allows performing a process appropriate for a plurality of types of sensors at various locations according to on-site needs.

According to the embodiment, the evaluation target image includes the behavior information. Therefore, it is possible to achieve an evaluation based on a surface posture of the subject 3 that is different depending on the sensor 2 and the measurement condition of the subject 3. This allows further improvement of the accuracy of evaluating the state of the subject 3, and allows performing a process appropriate for a plurality of types of sensors at various locations according to on-site needs.

According to the embodiment, since the evaluation result associated with the type of sensor 2 of the subject 3 and the specifications and the arrangement condition of the sensor 2 can be obtained, it is possible to identify the condition that causes the physical abnormality of the subject 3, and to grasp a change in the body due to a change in an observation condition and the like. This can lead to improvement of the body of the subject 3, a reduction in a burden of nursing care, and the like.

According to the embodiment, the evaluation target image includes the feature information. Therefore, it is possible to achieve an evaluation based on the feature of the factor of the measurement that is different depending on the type and the property of the sensor 2, the number of the sensors 2, and the arrangement of the sensor 2 and the like. This allows further improvement of the accuracy of evaluating the state of the subject 3, and allows performing a process appropriate for a plurality of types of sensors at various locations according to on-site needs.

According to the embodiment, the association is constructed by machine learning using the past evaluation target image and the reference information as learning data. Therefore, even when an unknown evaluation target image different from the past evaluation target image is evaluated, a quantitative evaluation can be performed. This allows attempting further improvement of the evaluation accuracy.

According to the embodiment, when the updating unit 16 newly acquires a relationship between the past evaluation target image and the reference information, the updating unit 16 reflects the relationship in the association. This allows the association to be easily updated, and thus attempting continuous improvement of the evaluation accuracy.

According to the embodiment, the evaluation means S130 refers to the reference database and generates the evaluation result for the evaluation target image. The reference information includes information on the body. Therefore, it is possible to generate the evaluation result based on the result of evaluating the state of the subject 3 in the past. This allows attempting improvement of the accuracy of evaluating the state of the subject 3.

While the embodiments of the present invention have been described, the embodiments have been presented as examples, and are not intended to limit the scope of the invention. These novel embodiments can be embodied in a variety of other configurations. Various omissions, substitutions and changes can be made without departing from the gist of the invention. The embodiments and the modifications thereof are within the scope and the gist of the invention and within the scope of the inventions described in the claims and their equivalents.

### DESCRIPTION OF REFERENCE SIGNS

1: Subject evaluation device
2: Sensor
2a to 2f: Sensor
3: Subject
4: Communications network
5: Another terminal
6: Server
10: Housing
11: Acquisition unit
12: Conversion unit
13: Evaluation unit
14: Output unit
15: Storing unit
16: Updating unit
100: Subject evaluation system
101: CPU
102: ROM
103: RAM
104: Storage unit
105: I/F
106: I/F
107: I/F
108: Input unit
109: Display unit
110: Internal bus
200a: Sensor data
201a: Sensor data
200b: Evaluation target image
201b: Evaluation target image
202b: Evaluation target image
A: Location
B: Location
S110: Acquisition means
S120: Conversion means
S130: Evaluation means
S140: Output means

1. A subject evaluation device that evaluates a state of a subject, comprising:
one or more sensors that measure the state of the subject;
an acquisition unit that acquires subject information indicating at least any of a physical condition and behavior of the subject, and feature information indicating a feature of the sensor via the sensor;
a conversion unit that converts the acquired subject information into an evaluation target image on a two-dimensional plane based on the feature information on the sensor;
a reference database that stores an association between a past evaluation target image that has been preliminarily converted and reference information associated with the past evaluation target image;
an evaluation unit that refers to the reference database and generates an evaluation result for the evaluation target image; and
an output unit that outputs the evaluation result.

2. The subject evaluation device according to claim 1, wherein
the association is constructed by machine learning using the past evaluation target image and the reference information as learning data.

3. The subject evaluation device according to claim 1, further comprising
an updating unit that reflects a relationship between the past evaluation target image and the reference information in the association when the relationship is newly acquired.

4. A subject evaluation system that evaluates a state of a subject, comprising:
one or more sensors that measure the state of the subject;
acquisition means that acquires subject information indicating at least any of a physical condition and behavior of the subject, and feature information indicating a feature of the sensor via the sensor;
conversion means that converts the acquired subject information into an evaluation target image on a two-dimensional plane based on the feature information on the sensor;
a reference database that stores an association between a past evaluation target image that has been preliminarily converted and reference information associated with the past evaluation target image;
evaluation means that refers to the reference database and generates an evaluation result for the evaluation target image; and
output means that outputs the evaluation result.

5. The subject evaluation device according to claim 2, further comprising
an updating unit that reflects a relationship between the past evaluation target image and the reference information in the association when the relationship is newly acquired.
